(19) United States
(12) Patent Application Publication
(10) Pub. No.: US 2023/0270765 A1
(43) Pub. Date: Aug. 31, 2023

(54) APOTHECARIAL COMPOSITIONS AND RELATED METHODS OF TREATMENT

(71) Applicant: Powderpost LLC, LAMESA, TX (US)

(72) Inventors: CURTIS CRISP, LAMESA, TX (US); JAY MOON, WOODLAND HILLS, CA (US)

(21) Appl. No.: 18/040,022

(22) PCT Filed: Aug. 2, 2021
(86) PCT No.: PCT/US2021/044200

§ 371 Date: Jan. 31, 2023

Related U.S. Application Data
(60) Provisional application No. 63/060,106, filed on Aug. 2, 2020.

(51) Int. Cl. A61K 31/00; A61K 31/351; A61P 17/02; A61K 9/00; A61K 9/107

(52) U.S. Cl. CPC A61K 31/658; A61K 31/351; A61P 17/02; A61K 9/0014; A61K 9/1075

(57) ABSTRACT

The present invention provides apothecarial compositions and treatments for medical conditions and/or for cosmetic applications.

### CLAIM OF PRIORITY UNDER 35 U.S.C. § 119

The present Application for Patent claims priority to Provisional Application No. 63/060,106 entitled “APOTHECARIAL COMPOSITIONS AND RELATED METHODS OF TREATMENT” filed Aug. 2, 2020, and assigned to the assignee hereof and hereby expressly incorporated by reference herein.

### BACKGROUND

The present invention discloses apothecarial compositions useful for treatment or prevention of medical conditions and/or for cosmetic applications, and treatment methods thereof.

### SUMMARY

Some embodiments of the invention relate to a composition including a nano-emulsified cannabinoid and amygdalin in a formulation suitable for treating or preventing a medical condition. In some embodiments, the cannabinoid is CBDa, CBC, CBG, or CBD. In some embodiments, the cannabinoid and the amygdalin act synergistically.

In some embodiments, the medical condition can be bed sores, sunburn, pain, rashes, ringworm, eczema, psoriasis, age spots, rosacea, acne, scarring, warts, shingles flare-ups, multiple sclerosis, viral infections, and/or the like.

Some embodiments of +the invention relate to a formulation including the composition combined with a base formulation for administration to a subject in a topical manner.

Some embodiments of the invention relate to a method for treating a patient with a medical condition. In some embodiments, the method can include administering the formulation to the patient, wherein administration results in the treatment or prevention of the medical condition. In some embodiments, the medical condition can be selected from bed sores, sunburn, pain, rashes, ringworm, eczema, psoriasis, age spots, rosacea, acne, scarring, warts, shingles flare-ups, multiple sclerosis, viral infections, and/or the like.

### DETAILED DESCRIPTION

Apothecarial compositions and methods for treatment or prevention of medical conditions and/or for cosmetic applications are provided. The invention also relates to methods for producing the compositions.

The composition can include amygdalin (also called nitriloside, purasin, and/or vitamin B17) or derivatives or variations thereof. For example, the composition can include Laetrile, a drug containing purified amygdalin. Amygdalin is found in many plants, such as in the seeds (kernels) of apricots, bitter almonds, apples, peaches, and plums. Thus, the composition can include extracts of plants containing amygdalin such as apricot seed extract. In some embodiments, apricot seed powder (ASP) with 50% amygdalin can be used. In other embodiments, percentage of amygdalin in the ASP can be greater or lower than 50% such as, for example, 1%, 2%, 5%, 10%, 20%, 30%, 40%, 60%, 70%, 80%, 90%, 95%, or 99%.

In some embodiments, the composition includes an oil. The oil can be a hydrocarbon-based oil that is in liquid form at room temperature and/or body temperature. In other embodiments, the oil can be solid or semi-solid at room temperature and/or at body temperature. Non-limiting examples of oils include plant-based oils, mineral oils, triglycerides, essential oils, and animal-based oils. In some embodiments, the oil is selected from the group consisting of plant-based oil, mineral oil, triglyceride, essential oil, and animal-based oil. In other embodiments, the oil is selected from the group consisting of light mineral oil, MCT oil, hydrogenated castor oil, jojoba oil, peppermint oil, cannabis oil, and/or the like.

In some embodiments, the composition includes a nano emulsion of a cannabinoid or derivative thereof. The cannabinoid can be, for example, one or more of cannabigerolic acid (CBGA), cannabigerolic acid monomethylether (CBGAM), cannabigerol (CBG), cannabigerol monomethylether (CBGM), cannabigerovarinic acid (CBGVA), cannabigerovarin (CBGV), cannabichromenic acid (CECA), cannabichromene (CBC), cannabichromevarinic acid (CBCVA), cannabichromevarin (CBCV), cannabidiolic acid (CBDA), cannabidiol (CBD), cannabidiol monomethylether (CBDM), cannabidiol-C4 (CBD-C4), cannabidivarinic acid (CBDVA), cannabidivarin (CBDV), cannabidiorcol (CBD-C1), delta-9-tetrahydrocannabinolic acid A (THCA-A), delta-9-tetrahydrocannabinolic acid B (THCA-B), delta-9-tetrahydrocannabinol (THC), delta-9-tetrahydrocannabinolic acid-C4 (THCA-C4), delta-9-tetrahydrocannabinol-C4 (THC-C4), delta-9-tetrahydrocannabivarinic acid (THCV A), delta-9-tetrahydrocannabivarin (THCV), delta-9-tetrahydrocannabiorcolic acid (THCA-C1), delta-9-tetrahydrocannabiorcol (THC-C 1), delta-7-cis-iso-tetrahydrocannabivarin, delta-8-tetrahydrocannabinolic acid (118-THCA), delta-8-tetrahydrocannabinol (118-THC), cannabicyclolic acid (CBLA), cannabicyclol (CBL), cannabicyclovarin (CBLV), cannnabielsoic acid A (CBEA-A), cannabielsoic acid B (CBEA-B), cannabielsoin (CBE), cannabinolic acid (CENA), cannabinol (CBN), cannabinol methylether (CBNM), cannabinol-C4 (CBN-C4), cannabivarin (CBV), cannabinol-C2 (CBN-C2), cannabiorcol (CBN-C1), cannabinodiol (CBND), cannabinodivarin (CB VD), cannabitriol (CBT), 10-ethoxy-9-hydroxy-delta-6a-tetrahydrocannabinol, 8,9-dihydroxy-delta-6a-tetrahydrocannabinol, cannabitriolvarin (CBTV), ethoxy-cannabitriolvarin (CB TVE), dehydrocannabifuran (DCBF), cannabifuran (CBF), cannabichromanon (CBCN), cannabicitran (CBT), 10-oxo-delta-6a-tetrahydrocannabinol (OTHC), delta-9-cis-tetrahydrocannabinol (cis-THC), 3,4,5,6-tetrahydro-7-hydroxy-alpha-alpha-2-trimethyl-9-n-propyl-2,6-methano-2H-1-benzoxocin-5-methanol (OH-iso-HHCV), cannabiripsol (CBR) and trihydroxy-delta-9-tetrahydrocannabinol (triOH-THC), or the like. In some embodiments, the cannabinoid is CBDA, CBC, CBG, or CBD.

In some embodiments, the composition can include a terpene. The terpene can be, but is not limited to, a-bisabolol, borneol, camphene, camphor, 3-carene, caryophyllene oxide, b-caryophyllene, a-cedrene, citronellol, p-cymene, eucalyptol, fenchol, geraniol, geranyl acetate, guaiol, a-humulene, isobomeol, (−)-isopulegol, limonene, linalool, menthol, myrcene, nerolidol, ocimene, phellandrene, phytol, a-pinene, b-pinene, R-(+)-pulegone, sabinene, a-terpinene, terpinen-4-ol, a-terpineol, 4-terlineol, terpinolene, valencene, or the like.

The term, “nanoparticle” or “nano emulsion” in the present disclosure refers to different types of compositions or nano-scale particles as carriers that encapsulate or contain one or more nutraceutical supplements, by using a molecular assembly technique to carry the nutraceutical supplements across biological barriers, such as skin, to deliver the nutraceutical factors to target cell sites of the human body where they are released. Lipid nanoparticles may be or include those less than 100 nm in diameter, with the average size 1 nm to 500 nm in some embodiments. In some embodiments, the average size is less than 50 nm. In some embodiments the average size of the nanoparticles can be 2 nm, 5 nm, 10 nm, 20 nm, 30 nm, 40 nm, 60 nm, 70 nm, 80 nm, 90 nm, 100 nm, 125 nm, 150 nm, 175 nm, 200 nm, 250 nm, 300 nm, 350 nm, 400 nm, 450 nm, or the like.

In some embodiments, the particle can be an angstrom-sized cannabinoid or an angstrom-sized cannabinoid-containing particle. As used herein, angstrom-sized emulsions are considered to be nano emulsions.

In some embodiments, the composition includes hyaluronic acid or derivatives or variations thereof. Hyaluronic acid is a disaccharide polymer formed from D-glucuronic acid and N-acetyl-glucosamine molecules. Hyaluronic acid is naturally present in many tissues, mainly the skin, particularly the epidermis, and even in connective tissues and is one of the main components of the extracellular matrix. The length of the molecule depends on the tissue, species and tissue condition. Hyaluronic acid can be obtained by tissue extraction from animal tissues or, inter alia, by bacterial fermentation using Streptococcus equi or Bacillus subtilis.

In some embodiments, the composition can include a viscosity-controlling agent/emulsifier. Suitable emulsifiers can be, for example, hydrogenated lecithin, glycerin, sodium gluconate, acrylates, CIO-30 alkyl acrylate crosspolymer, sodium carboxymethyl betaglucan, castor oil, polyglyceryl-3 methylglucose distearate, cetearyl alcohol, behenyl alcohol, butylene glycol, propylene glycol, xanthan gum, potassium cetyl phosphate, polyglyceryl-6 distearate jojoba esters, polyglyceryl-3 beeswax, cetyl alcohol, PEG-800, laureth-7, C13-14 isoparrafin or other parrafins or isoparrafins, polyisobutene, sodium carboxymethyl betaglucan, PEG-200 hydrogenated glyceryl palmate, cellulose gum, PEG-7 glyceryl cocoate, aluminum starch octenylsuccinate, and/or the like.

In some embodiments, the composition can include a solvent. These solvents can include, for example, acetone dichloromethane, acetonitrile, n-butyl ether, monomethylacetamide, dipropylene glycol monomethyl ether, diethyl phthalate fatty acid esters, such as the diethyl ester or diisobutyl adipate, water, alkanol, benzyl benzoate, dipropylene glycol monomethyl ether, diethylene glycol monobutyl ether, silicone, dimethylacetamide, 2,2-dimethyl-4-oxy-methylene-1,3-dioxolane. N,N-dimethylalkanamides (e.g. N,N dimethylformamide), limonene, eucalyptol, dimethyl sulfoxide (DSMO), alkylpyrrolidones (e.g. N-methylpyrrolidone, 2-pyrrolidone), liquid polyoxyethylene glycols, methylene glycol, ethylene glycol, propylene glycol, dipropylene glycol, polypropylene glycol, butyl diglycol, dipropylene glycol, propylene carbonate, butylene carbonate, paraffins (e.g., white mineral oils, normal paraffins, isoparaffins), alkylbenzenes, alkylnaphthalenes, glycerine, glycerol triacetate, sorbitol, triacetin, aromatic hydrocarbons, dearomatized aliphatics, alkylbenzenes, alkylnaphthalenes, ketones such as methyl ethyl ketone, cyclohexanone, 2-heptanone, isophorone and 4-hydroxy-4-methyl-2-pentanone, acetates such as ethyl acetate, benzyl acetate, isoamyl acetate, hexyl acetate, heptyl acetate, octyl acetate, nonyl acetate, tridecyl acetate and isobornyl acetate, other esters such as alkylated lactate esters, dibasic esters and γ-butyrolactone, and alcohols, which can be linear, branched, saturated or unsaturated, such as phenyl ethyl alcohol, methanol, ethanol, n-propanol, isopropyl alcohol, n-butanol, isobutyl alcohol, n-hexanol, 2-ethylhexanol, n-octanol, decanol, isodecyl alcohol, isooctadecanol, cetyl alcohol, lauryl alcohol, tridecyl alcohol, oleyl alcohol, cyclohexanol, tetrahydrofurfuryl alcohol, diacetone alcohol and benzyl alcohol. Such solvents also include glycerol esters of saturated and unsaturated fatty acids (typically C6-C22), such as plant seed and fruit oils (e.g., oils of olive, castor, linseed, sesame, corn (maize), peanut, sunflower, grapeseed, safflower, cottonseed, soybean, rapeseed, coconut and palm kerne and mixtures thereof, e.g., polyethoxylated castor oil. Such solvents also include alkylated fatty acids (e.g., methylated, ethylated, butylated) wherein the fatty acids may be obtained by hydrolysis of glycerol esters from plant and animal sources, and can be purified by distillation. In some embodiments, the solvent is DSMO.

In some embodiments, the composition can further include an anti-irritation agent, anti-oxidant, terpene, cannabis terpene, anti-skin-redness agent, anti-adherent, binder, coating, disintegrant, flavor, color, lubricant, glidant, sorbent, preservative, filler, emulsifier, humectant, thickener, skin nourishing agent, skin moistening agent, occlusive agent, emollient agent, calming agent, natural smell agent, suspending agent, soothing agent, pH adjustment agent, complexant, purified water and/or the like and/or any combination thereof. For example, the composition can include an essential oil, collagen, lanolin, and/or the like and/or any combination thereof.

In some embodiments, the composition includes a mixture of at least one cannabinoid and a solvent, such as DSMO. This mixture can be added to a base formulation to form a formulation. The base formulation can be body butter cream, facial cream, deep moisture night cream, massage lotion cream, hand and body cream, hand soap cream, hand soap base, shampoo base, pet shampoo base, sugar/salt scrub oil base, heel and foot lotion cream, massage oil base, shower gel base, liquid hand soap base, milk bath base, hair and beard oil base, and/or the like. For example, the composition can be added to a face cream base that includes collagen and lanolin. A “cream base,” as used herein, can be any lotion or cream that is capable of being applied topically to a subject. Likewise, a “shampoo base,” as used herein, can be any formulation that is capable of being used as a shampoo. The amygdalin can be added to the base formulation.

For example, in some embodiments the cream base can include one or more of hyaluronic acid, water, isopropyl palmitate, propylene glycol, glyceryl stearate, isononyl isonanoate, glycerin, lanolin oil, myristyl myristate, stearic acid, carbomer, methylparaben, diazolidinyl urea, iodopropynyl butylcarbamate, disodium EDTA, allantoin, triethanolamine, sorbitan stearate, polysorbate, dimethicone, propylparaben, Aloe barbadensis leaf juice, retinyl palmitate, vitamin D, tocopheryl acetate, and/or the like.

In some embodiments, the composition provides a synergistic effect with respect to treatment/inhibition of medical conditions or achievement of desired cosmetic effects as compared to the effect provided by the components of the composition administered separately in similar concentrations.

In some embodiments, the composition includes at least two active agents, which can act synergistically. Table 1 provides non-limiting examples of such combinations. For example, Formula 1 includes amygdalin and CBD. For example, Formula 2 includes amygdalin and CBDa. For example, Formula 3 includes amygdalin and CBC. For example, Formula 4 includes amygdalin and CBG.

TABLE 1
Synergistic pairwise combinations

| | Amygdalin |
|---|---|
| CBD | Formula 1 |
| CBDA | Formula 2 |
| CBC | Formula 3 |
| CBG | Formula 4 |

The ratio of the synergistic ingredients can be about 1:1, 1:2, 1:3, 1:6, 1:9, 1:12, 2:1, 2:3, 2:9, 3:1, 3:2, 3:12, 6:1, 9:1, 9:2, 9:12, 12:1, 12:3, 12:9, or more or less.

Table 2 lists major ratios of pairwise combinations of synergistic active agents of the invention, named as ratios A through S where the first active agent is 51 and the second active agent is S2. For example, 51 can be amygdalin and S2 can be a cannabinoid. While the table provides a range of ratios that can be useful, it is within the scope of the invention to provide formulations in which synergistic ratios are adapted to particular uses.

TABLE 2

| | | Active Agent 2 | | | | | | | | | | | |
|---|---|---|---|---|---|---|---|---|---|---|---|---|---|
| | | 1 part S2 | | 2 parts S2 | | 3 parts S2 | | 6 parts S2 | | 9 parts S2 | | 12 parts S2 | |
| Active Agent 1 | 1 part S1 | 1:1 | (A) | 1:2 | (G) | 1:3 | (J) | 1:6 | (M) | 1:9 | (N) | 1:12 | (Q) |
| | 2 parts | 2:1 | (B) | 2:2 | (A) | 2:3 | (K) | 2:6 | (J) | 2:9 | (O) | 2:12 | (M) |
| | S1 | | | | | | | | | | | | |
| | 3 parts | 3:1 | (C) | 3:2 | (H) | 3:3 | (A) | 3:6 | (G) | 3:9 | (J) | 3:12 | (R) |
| | S1 | | | | | | | | | | | | |
| | 6 parts | 6:1 | (D) | 6:2 | (C) | 6:3 | (B) | 6:6 | (A) | 6:9 | (K) | 6:12 | (G) |
| | S1 | | | | | | | | | | | | |
| | 9 parts | 9:1 | (E) | 9:2 | (I) | 9:3 | (C) | 9:6 | (H) | 9:9 | (A) | 9:12 | (S) |
| | S1 | | | | | | | | | | | | |
| | 12 parts | 12:1 | (F) | 12:2 | (D) | 12:3 | (L) | 12:6 | (B) | 12:9 | (P) | 12:12 | (A) |
| | S1 | | | | | | | | | | | | |

Likewise, the synergistic formulations of the invention can include additional ingredients to further synergistically enhance the efficacy of a pairwise combination. In such embodiments, a paired combination is enhanced with a tertiary ingredient. In some embodiments, a quaternary ingredient. Likewise in other embodiments, additional synergists can be selected. The tertiary ingredient, quaternary ingredient or additional synergists can be a cannabinoid.

While embodiments of the invention can include active ingredients, inert ingredients, scents, and other formulation components, preferred embodiments begin with a primary blend. A primary blend is preferably a synergistic combination containing two or more active ingredients and, optionally, additional ingredients. The primary blends can then be combined with other ingredients to produce a final product. Accordingly, where concentrations, concentration ranges, or amounts, are given herein, such quantities can be in reference to a primary blend or blends. Thus, when a primary blend is further modified by addition of other ingredients to produce a formulation, the concentrations of the active ingredients are reduced proportional to the presence of other ingredients in the product. In some embodiments, the nutritional product can be capable of being used in combination with any food or beverage for human or animal consumption.

The terms “synergistic” and “synergistically effective” are used in the present invention to mean a biological effect created from the application of two or more agents that is greater than the sum of the biological effects produced by the application of the individual agents. Quantification of synergistic effects can be found in or adapted from S. R. Colby, “Calculating Synergistic and Antagonistic Response of Herbicide Combinations” Weeds 15(1): 20-23, 1967; the entire contents of the foregoing is fully incorporated by reference herein.

In some embodiments, the nano-emulsion of the cannabinoid can be between about 1-50% of the composition before adding to the base formulation. In some embodiments, the cannabinoid can be about 5-15%. For example, the nano-emulsion of the cannabinoid can be about 5, 6, 7, 8, 9, 10, 11, 12 13, 14, 15, 16, 17, 18, 19, 20, or more %. In some embodiments, the amygdalin can be between about 1-50% of the composition before adding to the base formulation. In some embodiments, the amygdalin can be about 1-20%. For example, the amygdalin can be about 1, 2, 3, 4, 5, 6, 7, 8, 9, 10, 11, 12 13, 14, 15, 16, 17, 18, 19, 20, or more %.

In some embodiments, the solvent can be between about 1-30% of the composition before adding to the base formulation. For example, the solvent, can be about 1, 2, 3, 4, 5, 6, 7, 8, 9, 10, 11, 12 13, 14, 15, 16, 17, 18, 19, 20, 21, 22, 23, 24, 25, 26, 27, 28, 29, 30, or more %.

In some embodiments, the oil can be about 15-35% of the composition before adding to the base formulation. For example, the castor oil can be about 10, 11, 12 13, 14, 15, 16, 17, 18, 19, 20, 21, 22, 23, 24, 25, 26, 27, 28, 29, 30, 31, 32, 33, 34, 35, or more %.

The formulation can have a “target dose” or “therapeutic dose”, which are terms that can be used interchangeably. The target dose is the amount of active ingredient that is targeted to be delivered to a user after application.

In some embodiments, the target does of amygdalin can be between 100 mg and 5000 mg. In some embodiments, the dose can be between 500 and 3000 mg amygdalin. For example, the dose can be about 500, 1000, 1500, 2000, 2500, or 3000 mg amygdalin.

In some embodiments, the target does of a cannabinoid can be between 1 mg and 2500 mg. In some embodiments, the dose can be between 500 and 5000 mg. For example, the dose can be about 500, 1000, 1100, 1200, 1300, 1400, 1500, 2000, or 2500, and 3000 mg cannabinoid.

In some embodiments, the composition can treat and/or inhibit dermatological conditions, such as hyperproliferative and/or inflammatory skin disorders, also inflammatory skin diseases. Diseases can include seborrheic dermatitis, lupus erythematosus, discoid lupus erythematosus, dermatomyositis, lichen planus, lichen sclerosis, psoriasis, lichen striatus, lichen aureus, granuloma faciale, atopic dermatitis, sweet syndrome, granuloma inguinale, pyoderma gangrenosum, necrobiotic xanthogranuloma. In some embodiments, the composition can be used for bed sores, sunburn, age spots, rashes, and/or the like. In some embodiments, the composition can be used for topical pain management.

In some embodiments, administration of the composition to a subject can relieve/improve erythema, redness, induration, thickness, desquamation, scaling, red patches of skin covered with silvery scales, small scaling spots, dry skin, cracked skin that may bleed, itching, burning, soreness, thickened, pitted or ridged nails, age spots, swollen and stiff joints, and any combination thereof.

In some embodiments, administration of the composition to a mammalian subject can improve the appearance of hair, fur, cowhide, skin, lips, and/or nails. Improving the appearance can be one of lightening of hair, skin, lips, and/or nails or by reducing, preventing, or treating inflammation of the skin or lips, including inflammation occasioned by ailment, affliction or disease of the skin or lips, or inflammation induced or inducible by an external agent. The external agent can be a virus. In some embodiments, the composition can improve the aesthetic appearance of skin. Improving the aesthetic appearance of the skin can include reduction in pore size; improvement in skin tone, radiance, clarity and/or tautness; promotion of anti-oxidant activity; improvement in skin firmness, plumpness, suppleness, and/or softness; improvement in procollagen and/or collagen production; improvement in skin texture and/or promotion of retexturization; improvement in skin barrier repair and/or function; improvement in appearance of skin contours; restoration of skin luster and/or brightness; replenishment of essential nutrients and/or constituents in the skin decreased by aging and/or menopause; improvement in communication among skin cells; increase in cell proliferation and/or multiplication; increase in skin cell metabolism decreased by aging and/or menopause; improvement in skin moisturization; promotion and/or acceleration of cell turnover; enhancement of skin thickness; reducing skin sensitivity; increase in skin elasticity and/or resiliency; and enhancement of exfoliation, with or without the use of alpha or beta hydroxy acids, keto acids or other exfoliants.

In some embodiments, the composition can be administered transdermally, topically, and/or the like.

Some embodiments of the invention relate to a formulation including the composition. The formulation can be in the form of a cream, ointment, lotion, foam, spray, shampoo, film, transdermal patch, and any combination thereof. In some embodiments the formulation can be adapted for internal use and can be delivered in the form of a suppository and/or solution for IV administration.

Some embodiments of the invention relate to methods of treating or preventing a medical condition including administering the composition or formulation to a subject in a therapeutically effective dosage, wherein the administration of the composition achieves any of the effects described in this disclosure. In some embodiments, the subject experiences at least one of (a) blocking of the disease process and avoidance of any disease symptoms; (b) interference with the disease process and (i) reduction of any disease symptoms and/or (ii) an accelerated recovery from any disease symptoms.

The medical condition can be, but is not limited to, rashes, pain, bed sores, dandruff, ringworms, eczema, psoriasis, age spots, rosacea, acne, scarring, warts, shingles flare ups. MS, viral infections, cancers, and/or the like. Cancers can include skin cancer.

Some embodiments of the invention relate to methods of cosmetic application of the composition or formulation to a subject in an effective dosage, wherein the administration of the composition achieves any of the effects described in this disclosure.

In some embodiments, the composition is administered once, twice, three, four times, or more through the day. In some embodiments, the composition is administered over a time period of about 1 day to about 6 months or more.

In some embodiments, the subject can be a human, large animal, or small animal.

Some embodiments of the invention relate to methods of producing the composition. The method can include heating an oil and/or nano-emulsion of a cannabinoid for a period of time. The cannabinoid can be heated at about 100-170 degrees F. For example, the cannabinoid can be heated at about 100, 110, 120, 130, 140, 150, or more degrees F. The period of time can be about 3, 4, or 5 min or more while increasing heat. In some embodiments, other ingredients, such as a viscosity-controlling agent/emulsifier DMSO, a solvent, etc. can be added into the solution. In some embodiments, Amygdalin can be added once the solution reaches about 100, 110, 120, 130, 140, or 150 degrees F. In some embodiments, the solution can be mixed at about 150 degrees to 155 degrees for a period of time. The period of time can be 1, 2, 3, 4, 5, 6, 7, 8, 9, 10, or more minutes. In some embodiments, the temperature of the solution can be kept under about 155, 156, 157, 158, 159, or 160 degrees F. The solution can be removed from heat and allowed to cool. For example, the solution can be allowed to cool to about 70, 80, 90, 100, 110, 120, or 130 degrees F.

In some embodiments, the cooled solution can be added to a base. Optionally, an essential oil scent can be added. The solution can be mixed for about 2, 3, 4, 5, 6, 7, 8, 9, 10, 11, 12, 13, 14, 15, or more minutes and then can be ready to package.

Further information can be found in PCT Application No.: PCT/US2019/062979, filed on Nov. 25, 2019, entitled “METHODS AND COMPOSITIONS FOR USE IN TREATMENT OF CANCER WITHOUT PSYCHOACTIVE EFFECTS,” which is incorporated by reference in its entirety herein.

### EXAMPLES

### Example 1

### Demonstration of Synergy

Experiments are done to demonstrate synergy of the components of the composition. Quantification of putative synergistic effects is based upon an adaptation of the Colby method (S. R. Colby, “Calculating Synergistic and Antagonistic Response of Herbicide Combinations” Weeds 15(1): 20-23, 1967). Specifically, each putative synergistic agent is administered to cells in an in vitro model and/or to animals in an animal model of the disease process, and the quantitative response is recorded. Then the combination of the active agents is made at 3:1, 1:1, and 1:3 ratios and each combination is contacted with the model assay system along a dilution gradient. The response of each treatment is quantified and a calculation of synergy is derived. Synergistic combinations in terms of ratios of the two components and in terms of dose-response are further optimized in subsequent tests based upon the first series of tests. Accordingly a synergistic formulation and dosing protocol is developed.

### Example 2

### Shampoo Formulations for Small Animals

Ingredients for 20 16 oz jars. 1500 mg amygdalin target dose:
30 g amygdalin
200 mL distilled H₂O
9129 mL Shampoo Base
Scent

### Example 3

### Topical Cream

Ingredients for 50 4 oz jars with a 1500 mg CBD target dose:
400 ML MCT oil
80 grams nano emulsified cannabinoid—for example CBD or CBDa or CBG or CBC oil
50 grams amygdalin (or B17, Laetrile, or apricot seed extract)
25 grams hyaluronic acid
25 grams of castor oil
100 mL DMSO (3% of solution before adding Face Cream Base)
4450 grams of a Collagen and Lanolin Face Cream Base

### Example 4

### Topical Cream for Large Animals

Ingredients for 20 16 oz jars. 1500 mg amygdalin target dose
30 grams amygdalin
200 mL distilled H₂O
9129 mL shampoo base
Scent

### Example 5

### Skin Cream

Ingredients of a skin cream
1500 mg amygdalin
CBDa
CBG
CBD

This formula can be used to treat acne, age spots, psoriasis, eczema, shingles and Molluscum

### Example 6

### Shampoo

Ingredients of a shampoo
1500 mg nanoemulsified CBDa, CBD, CBC, and CBG

This shampoo helps with itchy scalp, head sores, dandruff and fungus of the scalp. It can also promote hair growth, for example, can assisting in regrowing damaged hair in patients that have COVID-19.

### Example 7

4 oz 1500 mg nanoemulsified CBD target dose
Amygdalin
CBDa

This cream is for inflammation and muscle pains. For example, it has been used with patients suffering from joint pain. It is also used post-surgery to manage inflammation.

Patients diagnosed with a medical condition are treated with the composition. After treatment, patients demonstrate improved symptoms related to the medical condition, as set forth in the table below.

TABLE 3

| Original Medical | | # of Patients |
|---|---|---|
| Condition | Criteria for Improvement | Observed |
| Dandruff | Decreased itchiness, decreased | 8 |
| | flaking of skin | |
| Ringworms | Decreased number | 50 |
| Eczema | Decreased itchiness, decreased | 20 |
| | inflammation | |
| Psoriasis | Reduction | 15 |
| Age Spots | Lightening of color | 20 |
| Rosacea | Reduction | 4 |
| Acne | 75% reduction | 50 |
| Scarring | Softening and less visible | 20 |
| Warts | Decreased | 15 |
| Shingles | Pain relief | 2 |

### Example 8

### Method for Producing Composition

The following is a method for producing the product in Example 3. All temperature numbers are degrees Fahrenheit.

MCT oil and CBD oil is heated to 130 degrees for 5 min while increasing heat. As the heat is rising, the hyaluronic acid and the castor oil are added. DMSO is also added. Upon reaching 150 degrees, B 17 is added and mix. The B 17 will go into solution at 150 degrees. The solution is mixed at 150 degrees to 155 degrees for 5-10 minutes. The solution is removed from heat and allow the oil to cool back down to 130 degrees.

While the oil is cooling, the face cream base is measured and placed in a mixing bowl. Once the cream is in the bowl and the oil has cooled back to 130 degrees, the oil is added to the bowl and mixed. Optionally, 3 ML of an essential oil scent is add to the mixing cream. The solution is mixed for about 15 minutes and is then ready to package.

### Example 9

The following table provides exemplary cream-based topical formulations and with target doses:

TABLE 4

| | “Crème de la cream” | “Wreak Repair” | “Xtreme Cream THC” | “Xtreme Cream CGB” |
|---|---|---|---|---|
| Target dose | 1100 mg CBDa CBD | 1500 mg CBDa CBD | 1500 mg CBDa CBD | 1500 mg CBDa CBD |
| cannabinoid | 1100 mg delta 8 THC | | 1500 mg delta 8 THC | 1100 mg CGB |
| Target dose | 1500 mg | 1500 mg | 1500 mg | 1500 mg |
| amygdalin | (amygdalin) | Amygdalin | Amygdalin | Amygdalin |
| | 3000 mg ASP | | | |
| Other | DMSO | Cream base, | Cream base | Cream base |
| | Cream base | Castor oil | Castor oil | Castor oil |
| | Castor oil | | | |
| terpenes | “Tequila shot | “Tequila shot | “Tequila shot | “Tequila shot |
| | terpene profile” | terpene profile” | terpene profile” | terpene profile” |

### Example 10

The following table provides exemplary cream-based topical formulations and with amounts to produce approximately 125-130 4 oz jars.

TABLE 5

| | “Crème de la cream” | “Wreak Repair” | “Extreme Cream CBG” | “Extreme Cream THC” |
|---|---|---|---|---|
| Cannabinoid | 75 g CBDa | 115 g CBDa | 115 g CBDa | 115 g CBDa |
| | 45 g CBD | 85 g CBD | 85 g CBD | 85 g CBD |
| | 117 g delta-8 THC | | 117 g CBG | 117 g delta-8 THC |
| Amygdalin | 375 g Apricot Seed | 375 g Apricot Seed | 375 g Apricot Seed | 375 g Apricot Seed |
| | Powder | Powder | Powder | Powder |
| Other | 870 mL DSMO | 800 mL DSMO | 800 mL DSMO | 800 mL DSMO |
| | 12,738 g cream base | 12,855 g cream base | 12,738 g cream base | 12,738 g cream base |
| | 180 mL Castor oil | 180 mL Castor oil | 180 mL Castor oil | 180 mL Castor oil |
| Terpenes | 25 mL “Tequila shot | 25 mL “Tequila shot | 25 mL “Tequila shot | 25 mL “Tequila |
| | terpene profile” | terpene profile” | terpene profile” | shot terpene profile” |
| Scent | | 100 g Frankincense | 100 g Frankincense | 100 g Frankincense |
| | | 100 g Flawlwss | 100 g Flawlwss | 100 g Flawlwss |

### Example 11

The following protocol is used to produce “Wreck Repair” of Examples 9 and 10.

TABLE 6

| Step | Action |
|---|---|
| 1 | Factoring in a 2% loss, the following formulation will |
| | create approximately (125-130) 4 oz jars. Always wear |
| | hair net, gloves, mask and maintain a sterile, clean |
| | environment. |
| | HEAT ADDED IS 3 GRAMS PER 400 GRAMS OF CREAM |
| 1.1 | Using the water bath heat 115 g of CBDa (color |
| | remediated if possible) 85 grams CBD raw oil hemp oil. |
| | Maintain temperatures not to exceed 135°. Once the |
| | material is at temp combine into a glass beaker |
| | adding 800 ML's of Pharma grade DMSO. Maintain |
| | temperature mixing for 30 minutes to drop hemp oil |
| | into solution. |
| | Wait until the hemp oil solution cools to around |
| | 100° before adding to the cream base. |
| 1.2 | Add 12,855 grams of cream base into the overhead mixing bowl. |
| | Add 180 ML's of Castro oil. |
| | Add CBD delta-8 or CBG DMSO oil mixture to the |
| | cream base mixing bowl and begin to run at LOW |
| | speed for 30 minutes. Hand mix oil into cream before |
| | turning mixer on. |
| | Occasionally stop the mixer lower the bowl, scrape |
| | the sides to make sure all of the oil solution |
| | is fully homogenized into the cream base. |
| | At 25 mL's of tequila shot terpene profile. |
| 1.3 | Add 100 grams Frankincense, 100 grams Flawlwss, |
| | Once the cream base and oil solutions are fully |
| | homogenized add 375 grams of apricot seeds powder (50% |
| | amygdalin) into the mixer using a flour sifter. Run |
| | at LOW speed until all of the powder is mixed in |
| | with the cream base. Once all the ingredients |
| | are homogenized run at MED speed for 45 minutes. |

### Example 12

The following protocol is used to produce “Crème de la Cream” of Examples 9 and 10.

TABLE 7

| Step | Action |
|---|---|
| 1 | Factoring in a 2% loss, the following formulation will |
| | create approximately (98) 4 oz jars. Always wear hair net, |
| | gloves, mask and maintain a sterile, clean environment. |
| | HEAT ADDED IS 3 GRAMS PER 400 GRAMS OF CREAM |
| 1.1 | Using the water bath heat 75 g of CBDa (color |
| | remediated if possible) 45 grams CBD raw oil in |
| | addition to 117 g of delta-8 THC hemp oil. Maintain |
| | temperatures not to exceed 135°. Once the |
| | material is at temp combine into a glass |
| | beaker adding 870 ML's of Pharma grade DMSO. |
| | Maintain temperature mixing for 30 minutes to |
| | drop hemp oil into solution. |
| | Wait until the hemp oil solution cools to |
| | around 100° before adding to the cream base. |
| 1.2 | Add 12,738 grams of cream base into the overhead mixing bowl. |
| | Add 180 ML's of Castro oil. |
| | Add CBD delta eight DMSO oil mixture to the cream |
| | base mixing bowl and begin to run at LOW speed for 30 |
| | minutes. Hand mix oil into cream before turning mixer on. |
| | Occasionally stop the mixer lower the bowl, scrape |
| | the sides to make sure all of the oil solution is |
| | fully homogenized into the cream base. |
| | At 25 mL's of tequila shot terpene profile. |
| 1.3 | Once the cream base and oil solutions are fully |
| | homogenized add 375 grams of apricot seeds powder |
| | into the mixer using a flour sifter. Run at LOW speed |
| | until all of the powder is mixed in with the cream base. |
| | Once all the ingredients are homogenized run at |
| | MED speed for 45 minutes. |

### Example 13

The following protocol is used to produce “Xtreme Cream” of Examples 9 and 10.

TABLE 8

| Step | Action |
|---|---|
| 1 | Factoring in a 2% loss, the following formulation will |
| | create approximately (125-130) 4 oz jars. Always |
| | wear hair net, gloves, mask and maintain a sterile, |
| | clean environment. |
| | HEAT ADDED IS 3 GRAMS PER 400 GRAMS OF CREAM |
| 1.1 | Using the water bath heat 115 g of CBDa (color |
| | remediated if possible) 85 grams CBD raw oil in |
| | addition to 117 g of CBG or delta-8 THC hemp oil. |
| | Maintain temperatures not to exceed 135°. |
| | Once the material is at temp combine into a glass |
| | beaker adding 800 ML's of Pharma grade DMSO. |
| | Maintain temperature mixing for 30 minutes |
| | to drop hemp oil into solution. |
| | Wait until the hemp oil solution cools to |
| | around 100° before adding to the cream base. |
| 1.2 | Add 12,738 grams of cream base into the overhead mixing bowl. |
| | Add 180 ML's of Castro oil. |
| | Add CBD delta-8 or CBG DMSO oil mixture to the cream |
| | base mixing bowl and begin to run at LOW speed for |
| | 30 minutes. Hand mix oil into cream before turning mixer on. |
| | Occasionally stop the mixer lower the bowl, scrape |
| | the sides to make sure all of the oil solution is |
| | fully homogenized into the cream base. |
| | At 25 mL's of tequila shot terpene profile. |
| 1.3 | Add 100 grams Frankincense, 100 grams Flawlwss, |
| | Once the cream base and oil solutions are fully |
| | homogenized add 375 grams of apricot seeds |
| | powder (50% amygdalin) into the mixer using |
| | a flour sifter. Run at LOW speed until all |
| | of the powder is mixed in with the cream base. |
| | Once all the ingredients are homogenized |
| | run at MED speed for 45 minutes. |

The various methods and techniques described above provide a number of ways to carry out the application. Of course, it is to be understood that not necessarily all objectives or advantages described are achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some embodiments specifically include one, another, or several features, while others specifically exclude one, another, or several features, while still others mitigate a particular feature by including one, another, or several other features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, any numbers expressing quantities of ingredients, properties such as molecular weight, reaction conditions, and so forth, used to describe and claim certain embodiments of the disclosure are to be understood as being modified in some instances by the term “about.” Accordingly, in some embodiments, the numerical parameters set forth in the written description and any included claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are usually reported as precisely as practicable.

In some embodiments, the terms “a” and “an” and “the” and similar references used in the context of describing a particular embodiment of the application (especially in the context of certain claims) are construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Variations on preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

What is claimed is:

1. A composition comprising a nano-emulsified cannabinoid and amygdalin in a formulation suitable for treating or preventing a medical condition; wherein the cannabinoid is CBDa, CBC, CBG, or CBD; wherein the cannabinoid and the amygdalin act synergistically.

2. The composition of claim 1, wherein the medical condition is selected from bed sores, sunburn, pain, rashes, ringworm, eczema, psoriasis, age spots, rosacea, acne, scarring, warts, shingles flare flare-ups, multiple sclerosis, and viral infections.

3. A formulation comprising the composition of claim 1, combined with a base formulation for administration to a subject in a topical manner.

4. A method for treating a patient with a medical condition comprising administering the formulation of claim 3 to the patient, wherein administration results in the treatment or prevention of the medical condition; wherein the medical condition is selected from bed sores, sunburn, pain, rashes, ringworm, eczema, psoriasis, age spots, rosacea, acne, scarring, warts, shingles flare flare-ups, multiple sclerosis, and viral infections.
